(19) United States
(12) Patent Application Publication
(10) Pub. No.: US 2021/0112837 A1
(43) Pub. Date: Apr. 22, 2021

(54) IMPROVED RECOVERY OF NITRATE REDUCTASE ACTIVITY

(71) Applicant: Chr. Hansen A/S, Hoersholm (DK)

(72) Inventors: Birgitte YDE, Hoersholm (DK); Tina Malling THORSEN, Hoersholm (DK); George Nabin BAROI, Hoersholm (DK); Robin TAPONEN, Hoersholm (DK); Jakob SOELTOFT-JENSEN, Hoersholm (DK)

(73) Assignee: Chr. Hansen A/S, Hoersholm (DK)

(21) Appl. No.: 17/051,852

(22) PCT Filed: May 3, 2019
(86) PCT No.: PCT/EP2019/061355

§ 371 Date: Oct. 30, 2020

(30) Foreign Application Priority Data
May 4, 2018 (EP) 18170840.5
Jul. 18, 2018 (EP) 18184181.8

(51) Int. Cl. A23L 13/40; C12N 1/20; C12N 9/06; A23L 5/41; A23B 4/22; A23L 33/135; A23L 33/175; A23L 33/18; A23L 33/19; A23L 33/185; A23L 33/145; A23L 33/125

(52) U.S. Cl. CPC A23L 13/45; A23Y 2220/65; C12N 9/0044; A23L 5/41; A23B 4/22; A23L 33/135; A23L 33/175; A23L 33/18; A23L 33/19; A23L 33/185; A23L 33/145; A23L 33/125; C12Y 107/99004; A23V 2002/00; C12N 1/20

(57) ABSTRACT

The present invention is related to the field of reddening of food products. In particular the present invention relates to the preservation of nitrate reductase activity of frozen and/or dried lactic acid bacteria culture or Micrococcaceae culture.

### FIELD OF THE INVENTION

The present invention is related to the field of reddening of food products and microbial cultures. In particular the present invention relates to the preservation of and/or increasing nitrate reductase activity of frozen and/or dried lactic acid bacteria cultures (e.g. one or more species of Staphylococcus) or Micrococcaceae cultures with the use of a survival enhancer and the use of said culture for reddening of meat products. Finally, the invention provides a method for increasing the nitrate reductase activity of a lactic acid bacteria and/or Micrococcaceae bacteria (such as e.g. a Staphylococcus strain) having nitrate reductase activity by use of a formulation comprising an amino acid, a peptide and/or a protein.

### BACKGROUND ART

Color formation and color stability are amongst the most critical quality traits of processed meat products and thus of great importance to the meat industry. The characteristic cured color can be derived from the concentration of heme pigments (myoglobin, hemoglobin), their chemical states and additives such as nitrogen oxides and reducing agents. In standard fermented meat products, such as salami, the characteristic cured color is a result of the chemical reaction between compounds derived from added nitrite/nitrate and the naturally occurring red myoglobin leading to the simultaneous formation of the bright red nitrosylmyoglobin, in which an axial ligand nitric oxide (NO) is coordinated to the central Fe²⁺ in heme.

Despite of all its desired properties (color formation, microbiologic safety), the safety of nitrite to human health has been questioned. Nitrite can cause the formation of unwanted compounds in cured meat, like N-nitrosamines which are questionable in regard to health. These compounds can be formed in principle due to the reaction of nitrite with secondary amines and amino acids in muscle proteins as well as in the gastrointestinal tract.

The use of species of lactic acid bacteria culture or Micrococcaceae culture for color formation is based on their ability of some strains to reduce inorganic nitrate to nitrite which is further degraded into the above described nitric oxide (NO), the active compound in the color formation process. Strains having high nitrate-reductase activity reported a significantly faster color formation in meat (Meat science 2008 April; 78(4): 492-501). The nitrate could be provided directly as nitrate salt or indirectly with a natural nitrate source (vegetable powders). Even if a nitrite salt is added, the addition of strains from the specified groups is recommended as nitrite is partially re-converted to nitrate.

Commercial starter cultures are commonly distributed as frozen cultures. At the low temperature the frozen cultures, most metabolic activities in the cell cease and cells can be maintained in this suspended, but viable, state for extended periods.

Concentrated frozen cultures are commercially very interesting since the cultures can be inoculated directly into the production container. By using concentrated frozen cultures the end-user avoids the otherwise obligatory, time-consuming intermediary fermentation step during which the starter culture is amplified, and the end-user reduces the risk of contamination significantly. Concentrated cultures may be referred to as DVS—direct vat Set™ cultures.

As an alternative to concentrated frozen cultures, concentrated freeze-dried DVS™ cultures may be prepared. These cultures have an additional advantage in that they can be shipped without refrigeration.

In general, possible damaging effects of freezing and thawing on the viability of living cells have been ascribed to cell dehydration and the formation of ice crystals in the cytosol during freezing.

An article by F. J. Chavarri et al. (Biotechnology letters, vol 10, 1, 11-16 (1988), “Cryoprotective agents for frozen concentrated starters from non-bitter Streptococcus Lactis strains”) describes the storage viability of a frozen pure Streptococcus lactis culture may be improved by addition of 5% lactose or 5% sucrose. The lactose or sucrose worked as cryoprotective agents. Streptococcus lactis is a former name of Lactococcus lactis subsp. lactis.

Similarly, an article by R. Cárcoba et al (Eur Food Res Technol (2000) 211, 433-437, “Influence of cryoprotectants on the viability and acidifying activity of frozen and freeze-dried cells of the novel starter strain Lactococcus lactis subsp. lactis CECT 5180”) describes that storage viability of a frozen pure Lactococcus lactis subsp. lactis culture could be improved by addition of different cryoprotective agents such as sugars (lactose, sucrose and trehalose), glutamic acid and gelatin.

The viability of freeze-dried cultures may also be improved by use of cryoprotective agents. For instance, EP0259739 describes a number of different cryoprotective agents for freeze-dried cultures. EP0259739 does not disclose agents for stabilization of Micrococcaceae bacteria, or species of the family Staphylococcaceae (such as species of the Staphylococcus genus).

A number of other prior art documents also describe the use of amino acids, peptides or proteins as cryoprotective agents (see US2001/008874, EP1867713, WO2013/186348, Champagne et al. 1991, and Mendoza et al. 2013). These do not disclose stabilization of Micrococcaceae bacteria, or species of the family Staphylococcaceae (such as species of the Staphylococcus genus).

Further, there are problems with loss of nitrate reductase activity during freezing and drying of bacteria such as Micrococcaceae bacteria or species of the family Staphylococcaceae (such as species of the Staphylococcus genus) even when using known cryoprotectants have been tested. So, there is a need for effective protectants that can be added to concentrated cultures used in the food industry, particularly the meat industry, for protection e.g. preserving or increasing the nitrate reductase activity of bacteria during freezing and drying. Improved stability of nitrate reductases during freezing and drying lead to improved color formation in food products.

### SUMMARY OF THE INVENTION

The inventors of the present invention have surprisingly found that a bacteria having nitrate reductase activity, such as lactic acid bacteria and Micrococcaceae bacteria cultures having nitrate reductase activity, in the presence of an amino acid, a peptide and/or a protein have increased stability and/or preservation of nitrate reductase activity during freezing and drying leading to an increased reddening when using the cultures for coloring of food products containing myoglobin. The example included herein demonstrate the effect of a formulation comprising an amino acid, a peptide or a protein on the nitrate reductase activity of a Staphylococcus specie and shows that the formulations of the invention is capable of preserving and/or increasing the nitrate reductase activity over a formulation only comprising carbohydrate.

Therefore, in a first aspect the present invention relates to a concentrated culture comprising lactic acid bacteria and/or Micrococcaceae bacteria (e.g. a Staphylococcus specie) having nitrate reductase activity and further comprising an amino acid, a peptide and/or a protein.

The second aspect of the present invention relates to a method for making a frozen concentrated culture comprising:
a) mixing a formulation comprising an amino acid, a peptide and/or a protein and lactic acid bacteria and/or Micrococcaceae bacteria (e.g. a Staphylococcus specie) having nitrate reductase activity;
b) freezing the resulting mixture of a) to obtain a frozen material; and
c) packing the frozen material.

The third aspect of the present invention relates to a method for making a dried concentrated culture comprising:
a) mixing a formulation comprising an amino acid, a peptide and/or a protein and lactic acid bacteria and/or Micrococcaceae bacteria (e.g. a Staphylococcus specie) having nitrate reductase activity;
b) drying the resulting mixture of a) to obtain a dried material;
c) optionally grinding the obtained dried material of b); and
d) packing the dried material.

The fourth aspect of the present invention relates to a method for making a freeze-dried concentrated culture comprising
a) mixing an amino acid, a peptide and/or a protein and lactic acid bacteria and/or Micrococcaceae bacteria (e.g. a Staphylococcus specie) having nitrate reductase activity;
b) freezing the resulting mixture of a) to obtain a frozen material;
c) subliming water from the frozen material by freeze drying the material of b); and,
d) packing the freeze-dried material of c).

The fifth aspect of the present invention relates to a method for reddening of a food product comprising the steps of
a) adding the concentrated culture of the present invention to a food product; and
b) fermenting, ripening or curing the food product with the concentrated culture of the present invention.

A sixth aspect of the present invention is directed to a food product comprising a composition according to the first aspect of the invention.

In a seventh aspect the present invention relates to the use of a composition according to the first aspect of the invention for reddening of a food product.

An eighth aspect of the invention relates to the use an amino acid, a peptide and/or a protein, for preserving and/or increasing the nitrate reductase activity of a bacteria selected from the group consisting of lactic acid bacteria and Micrococcaceae bacteria (such as a Staphylococcus specie).

A ninth aspect of the invention relates to a method for increasing the nitrate reductase activity of a bacteria having nitrate reductase activity selected from the group consisting of lactic acid bacteria and Micrococcaceae bacteria (such as e.g. a Staphylococcus strain) having nitrate reductase activity comprising the steps of:
a) mixing a formulation comprising an amino acid, a peptide and/or a protein and lactic acid bacteria and/or Micrococcaceae bacteria (such as e.g. a Staphylococcus specie) having nitrate reductase activity to obtain a mixture comprising an amino acid, a peptide and/or a protein and bacteria;
b) optionally freezing the resulting mixture of a) to obtain a frozen material;
c) drying the mixture of a) or the frozen material of step b) to obtain a dried material; and
d) optionally packing the frozen material obtained from step b) or the dried material of step c) to obtain a packaged product.

### DETAILED DESCRIPTION OF THE INVENTION

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention relates to a concentrated culture comprising a bacterium having nitrate reductase activity such as selected from the group consisting of lactic acid bacteria and Micrococcaceae bacteria (e.g. a Staphylococcus specie) and a formulation comprising an amino acid, a peptide and/or a protein.

The term “lactic acid bacteria and/or Micrococcaceae bacteria having nitrate reductase activity” as used herein refers to lactic acid bacteria and/or Micrococcaceae bacteria which is capable of converting nitrate to nitrite.

As used herein the term “lactic acid bacterium” (LAB) designates a gram-positive, microaerophilic or anaerobic bacterium which ferments sugars with the production of acids including lactic acid (as the predominantly produced acid), acetic acid and propionic acid. The industrially most useful lactic acid bacteria are mostly found among species of the order Lactobacillales, such as Lactococcus species (spp.), Streptococcus spp., Lactobacillus spp. Such as L. fermentum, L. pensosus and L. Plantarum, Leuconostoc spp., Pediococcus spp. Such as P. pentosaceus and P. acidilactici, Brevibacterium spp., Enterococcus spp. and Propionibacterium spp. Additionally, lactic acid producing bacteria belonging to the group of the strict anaerobic bacteria, bifidobacteria, i.e. Bifidobacterium spp. which are frequently used as food starter cultures alone or in combination with lactic acid bacteria, are generally included in the group of lactic acid bacteria. The lactic acid bacteria may be any lactic acid bacteria having nitrate reductase activity.

Although part of another family and order than Lactobacillales, even certain bacteria of the species Staphylococcus (e.g.: S. carnosus, S. equorum, S. sciuri, S. vitulinus and S. xylosus) have been referred to as LAB (Mogensen et al. (2002) Bulletin of the IDF No. 377, 10-19).

In a preferred embodiment of the present invention the Lactobacillus pentosus specie is the Lactobacillus pentosus strain CHCC4196 that was deposited with the Deutsche Sammlung von Mikroorganismen und Zellkulturen (DSMZ) under the accession no. DSM 25011.

Staphylococci are Gram-positive organisms that grow singly, in pairs, in chains, or in clusters. They are members of the family Staphylococcaceae, which in turn is of the order Bacillales, and the class Bacilli. The Staphylococcus specie may be any Staphylococcus specie having nitrate reductase activity such as e.g. a Staphylococcus vitulinus, a Staphylococcus carnosus or a Staphylococcus xylosus specie. Other Staphylococcus strains of interest are S. simulans, S. saprophyticus, S. lentus, S. pasteuri, S. sciuri, S. haemolyticus, S. warneri, S. equorum, S. cohnii, S. epidermidis, S. hominis, S. capitis, S. intermedius and S. succinus. Thus in one embodiment of the invention, the lactic acid bacteria having nitrate reductase activity is a specie selected from the group consisting of Staphylococcus vitulinus, Staphylococcus carnosus, Staphylococcus xylosus, S. simulans, S. saprophyticus, S. lentus, S. pasteuri, S. sciuri, S. haemolyticus, S. warneri, S. equorum, S. cohnii, S. epidermidis, S. hominis, S. capitis, S. intermedius and S. succinu and more preferably the lactic acid bacteria having nitrate reductase activity is a specie selected from the group consisting of Staphylococcus vitulinus, Staphylococcus carnosus, Staphylococcus xylosus.

In a preferred embodiment of the present invention the Staphylococcus specie having nitrate reductase activity is a Staphylococcus vitulinus specie, a Staphylococcus xylosus and/or a Staphylococcus carnosus specie having nitrate reductase activity.

In a more preferred embodiment the Staphylococcus specie having nitrate reductase activity is a Staphylococcus carnosus specie.

In a preferred embodiment of the present invention the Staphylococcus vitulinus specie is selected from the group consisting of the Staphylococcus vitulinus strain CHCC10896 that was deposited with the Deutsche Sammlung von Mikroorganismen und Zellkulturen (DSMZ) under the accession no. DSM 25789 and the Staphylococcus vitulinus strain CHCC11576 that was deposited with the Deutsche Sammlung von Mikroorganismen und Zellkulturen (DSMZ) under the accession no. DSM 27311 and mutants derived thereof. In another preferred embodiment of the present invention the Staphylococcus carnosus specie is a Staphylococcus carnosus strain CHCC4055 that was deposited with the Deutsche Sammlung von Mikroorganismen und Zellkulturen (DSMZ) under the accession no. DSM 32779 and mutants derived thereof.

Micrococcaceae is a family of bacteria containing gram-positive spheric cells that occur singly or in pairs, tetrads, packets, irregular masses, or even chains. Free-living, saprophytic, parasitic, and pathogenic species occur. The type genus is Micrococcus. The Micrococcaceae bacteria may be any Micrococcaceae bacteria having nitrate reductase activity. Particular Micrococcaceae bacteria of interest are species of the genus Kocuria.

Other species belonging to the family of Micrococcaceae are Actinomycetales. Acaricomes, Arthrobacter, Auritidibacter, Citricoccus, Enteractinococcus, Kocuria, Micrococcus, Nesterenkonia, Renibacterium, Rothia, Sinomonas, Yaniella and Zhihengliuella. Thus, in one embodiment of the invention, the bacteria having nitrate reductase activity is one or more species selected from the group consisting of Actinomycetales. Acaricomes, Arthrobacter, Auritidibacter, Citricoccus, Enteractinococcus, Kocuria, Micrococcus, Nesterenkonia, Renibacterium, Rothia, Sinomonas, Yaniella and Zhihengliuella. In a more preferred embodiment, the bacteria having nitrate reductase activity is one or more species selected from the group consisting of species of the genus Kocuria.

In a preferred embodiment of the present invention the Kocuria specie is a Kocuria salsicia. In a more preferred embodiment of the present invention the Kocuria specie is selected from the Kocuria salsicia strain CHCC5184 that was deposited with the Deutsche Sammlung von Mikroorganismen und Zellkulturen (DSMZ) under the accession no. DSM 32827, the Kocuria salsicia strain CHCC5185 that was deposited with the Deutsche Sammlung von Mikroorganismen und Zellkulturen (DSMZ) under the accession no. DSM 32828 and mutants derived thereof.

In a preferred embodiment of the present invention the concentrated culture comprises a Kocuria specie and/or a staphylococcus specie having nitrate reductase activity and a formulation comprising an amino acid, a peptide and/or a protein. Thus, in a preferred embodiment, the concentrated culture comprises a bacterium having nitrate reductase activity selected from the group consisting of Kocuria species and Staphylococcus species and a formulation comprising an amino acid, a peptide and/or a protein.

In the present context, the term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties (e.g. regarding nitrate reductase activity) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant less than 1%, less than 0.1, less than 0.01, less than 0.001% or even less than 0.0001% of the nucleotides in the bacterial genome have been replaced with another nucleotide, or deleted, compared to the mother strain.

In a preferred embodiment of the present invention the nitrate reductase activity of the specie is improved by at least 10%, such as at least 20%, such as at least 30%, such as at least 40%, such as at least 50% compared to a specie not being in the presence of an amino acid, peptide and/or a protein after freezing or drying treatment.

The percentage improvement of the nitrate reductase activity of a Staphylococcus specie in question can be readily determined by the determination of conversion rate of nitrate to nitrite (nitrate reductase activity) and by measuring the concentration of nitrite to a given time e.g. in a kinetic set-up of the Griess test (Reference to: Colorimetry and Spectrophotometry” Vogel's Textbook of Quantitative Chemical Analysis, 5th Edition. Longman. p. 702. ISBN 0-582-44693-7) as described in the Examples herein.

The formulation comprising an amino acid, a peptide and/or a protein may be only an amino acid, a peptide and/or a protein or it may comprise other ingredients.

The amino acids, peptides and/or proteins may be any amino acids, peptides and/or proteins.

The peptides and proteins may be from animal or vegetarian origin.

Particular proteins of interest include but are not limited to soy protein, wheat protein, pea protein, potato protein, cotton seed protein, skim milk powder, sodium caseinate, whey protein, meat protein and/or any hydrolyzed protein. In a preferred embodiment of the invention, the protein is selected from the group consisting of casein (or a salt thereof, such as sodium caseinate) and potato protein.

Particular peptides of interest include but are not limited to peptides derived from yeast, soy, wheat, pea, potato, cotton seed, skimmed milk, casein, whey, meat and/or any hydrolyzed protein. In one embodiment of the invention, the hydrolyzed protein is selected from the group consisting of hydrolyzed casein, hydrolyzed potato protein and hydrolyzed pea protein. Thus, in one embodiment of the invention, the peptides are selected from the group consisting of hydrolyzed casein, hydrolyzed potato protein, hydrolyzed pea protein and yeast extract.

In one embodiment of the invention, the formulation does not comprise any sort of gelatin, e.g. fish, beef or pork gelatin.

The amino acid is selected from the group consisting of glycine, alanine, valine, leucine, isoleucine, methionine, proline, phenylalanine, tryptophan, serine, threonine, cysteine, tyrosine, asparagine, glutamine, aspartic acid, glutamic acid, lysine, arginine and/or histidine. In a preferred embodiment of the invention, the amino acid is one or more amino acids selected from the group consisting of histidine, tryptophan, glycine and phenylalanine.

In one embodiment of the invention, the amino acid is histidine. In one embodiment of the invention, the amino acid is tryptophan. In one embodiment of the invention, the amino acid is glycine. In one embodiment of the invention, the amino acid is phenylalanine.

The amino acids and peptides may also be in the form of yeast extract.

In yet an embodiment of the invention the amino acid, peptide and/or protein is one or more species selected from the group consisting of casein, or a salt thereof such as sodium caseinate, potato protein, hydrolyzed casein, hydrolyzed potato protein, hydrolyzed pea protein, histidine, tryptophan, glycine, phenylalanine, and yeast extract. In such embodiments of the invention, one, two, three, or more species of said group may be selected.

In one embodiment of the invention, the concentrated culture comprises a formulation of one or more amino acids, peptides and/or proteins selected from the group consisting of casein, or a salt thereof such as sodium caseinate, potato protein, hydrolyzed casein, hydrolyzed potato protein, hydrolyzed pea protein, histidine, tryptophan, glycine, phenylalanine, and yeast extract, and lactic acid bacteria and/or Micrococcaceae bacteria (e.g. one or more species of Staphylococcus) having nitrate reductase activity.

The amount of proteins and/or peptides is at least 0.05% w/w based on the total composition before drying or freezing, such as at least 0.1%, 0.2%, 0.3%, 0.4%, 0.5% such as at least 0.6% based on the total composition before drying or freezing. The amount of proteins and/or peptides is at most 5% w/w, such as at most 4%, 3%, such as at most 2% based on the total composition before drying or freezing.

The amount of amino acids is at least 0.01% w/w based on the total composition before drying or freezing, such as at least 0.02%, 0.03%, 0.04%, 0.05% such as at least 0.06% based on the total composition before drying or freezing. The amount of amino acids is at most 1% w/w, such as at most 0.5%, 0.4%, 0.3%, 0.2%, such as at most 0.1% based on the total composition before drying or freezing.

The amount of proteins and/or peptides is at least 0.2% w/w based on the total composition after drying or freezing, such as at least 0.3%, 0.4%, 0.5%, 0.6%, 0.8%, 1%, 1.5%, 2% based on the total composition after drying or freezing. The amount of proteins and/or peptides is at most 20% w/w, such as at most 16%, 12%, 10%, 9%, 8%, 7%, 6%, 5% such as at most 4% based on the total composition after drying or freezing.

The amount of amino acids is at least 0.04% w/w based on the total composition after drying or freezing, such as at least 0.05%, 0.06%, 0.07%, 0.08%, 0.09%, 0.1%, 0.15%, 0.2% based on the total composition after drying or freezing. The amount of amino acids is at most 5% w/w, such as at most 4%, 3%, 2%, 1%, 0.5%, such as at most 0.4% based on the total composition after drying or freezing.

The concentrated culture of the present invention may further comprise a monosaccharide, a disaccharide, a trisaccharide, an oligosaccharide and/or a polysaccharide.

The disaccharide may be selected from but are not limited to the group consisting of sucrose, lactose and maltose. In a preferred embodiment the concentrated culture of the present invention further comprises sucrose. Thus, in a preferred embodiment of the invention, the concentrated culture comprises or consists of a formulation of an amino acid, a peptide and/or a protein and lactic acid bacteria and/or Micrococcaceae bacteria (e.g. one or more species of Staphylococcus) and a disaccharide (e.g. sucrose).

The polysaccharide may be maltodextrin. Maltodextrins are classified by DE (dextrose equivalent) and have a DE between 3 and 20. The higher the DE value, the shorter the glucose chains, the higher the sweetness, the higher the solubility, and the lower heat resistance. In a preferred embodiment the concentrated culture of the present invention further comprises maltodextrin which may be of a DE from 3 to 20 (e.g. maltodextrin DE 12). Thus, in a preferred embodiment of the invention, the concentrated culture comprises or consists of a formulation of an amino acid, a peptide and/or a protein and lactic acid bacteria and/or Micrococcaceae bacteria (e.g. one or more species of Staphylococcus) and a polysaccharide (e.g. maltodextrin).

In an even more specific embodiment of the invention the concentrated culture comprises or consists of a formulation of an amino acid, a peptide and/or a protein and lactic acid bacteria and/or Micrococcaceae bacteria (e.g. one or more species of Staphylococcus), a disaccharide (e.g. sucrose), a polysaccharide (e.g. maltodextrin) and optionally water.

In yet an even more specific embodiment of the invention the concentrated culture comprises or consists of a formulation of an amino acid, a peptide and/or a protein and lactic acid bacteria and/or Micrococcaceae bacteria (e.g. one or more species of Staphylococcus), sucrose and maltodextrin, and optionally water.

In yet an even more specific embodiment of the invention the concentrated culture comprises or consists of a formulation of an amino acid, a peptide and/or a protein and one or more species of Staphylococcus (e.g. a species of Staphylococcus carnosus), sucrose and maltodextrin, and optionally water.

The concentrated culture of the present invention may further comprise citric acid or a salt thereof (a citrate, such as for example a sodium salt of citrate). In a particular embodiment of the present invention the culture comprises a trisodium citrate.

The method for obtaining the concentrated culture of the present composition is by mixing a formulation of an amino acid, a peptide and/or a protein and lactic acid bacteria and/or Micrococcaceae bacteria (e.g. one or more species of Staphylococcus) having nitrate reductase activity. After mixing the composition may be frozen or dried.

In aspects wherein the mixture comprising amino acid, peptide and/or protein and bacteria is frozen, the freezing may be performed by allowing contacting the mixture with a cryogenic object, fluid or gas, e.g. by contacting the mixture with liquid nitrogen, such as e.g. by allowing the mixture to drip into liquid nitrogen, thereby forming pellets, threads or strings, wherein pellets are preferred. Alternatively, the mixture may be frozen in a tray en bloc and may thereafter be divided into smaller entities (e.g. pellets) or ground into smaller particles.

In aspects wherein the mixture comprising amino acid, peptide and/or protein, and a bacteria is dried to obtain a dried material, said drying may be performed using various techniques of the art, e.g. vacuum drying, spray drying, lyophilization, fluidized bed drying and/or desiccation. In a preferred embodiment, drying is performed by freeze drying or spray drying, and in a particular embodiment by freeze drying.

The method of drying may be but are not limited to vacuum drying, freeze drying and/or spray drying.

In a preferred embodiment the dried culture is obtained by
a) mixing a formulation of an amino acid, a peptide and/or a protein and lactic acid bacteria and/or Micrococcaceae bacteria (e.g. one or more species of Staphylococcus) having nitrate reductase activity and optionally other ingredients;
b) drying the resulting mixture of a) to obtain a dried material;
c) optionally grinding the obtained dried material of b); and
d) packing the dried material.

The drying may be performed by but are not limited to vacuum drying, freeze drying and/or spray drying.

In a more preferred embodiment of the present invention the freeze-dried culture is obtained by
a) mixing a formulation of an amino acid, a peptide and/or a protein and lactic acid bacteria and/or Micrococcaceae bacteria (e.g. one or more species of Staphylococcus) bacteria having nitrate reductase activity and optionally other ingredients;
b) freezing the resulting mixture of a) to obtain a frozen material;
c) subliming water from the frozen material to freeze-dry the material of b);
d) Optionally grinding the obtained freeze-dried material of c); and
e) packing the freeze-dried material.

In another preferred embodiment of the present invention a spray-dried culture is obtained by
a) mixing a formulation of an amino acid, a peptide and/or a protein and lactic acid bacteria and/or Micrococcaceae bacteria (e.g. one or more species of Staphylococcus) having nitrate reductase activity and optionally other ingredients;
b) spray drying the resulting mixture of a) to obtain a spray-dried material;
c) packing the spray-dried material.

In another preferred embodiment of the present invention a frozen culture is obtained by
a) mixing a formulation of an amino acid, a peptide and/or a protein and lactic acid bacteria and/or Micrococcaceae bacteria (e.g. one or more species of Staphylococcus) having nitrate reductase activity and optionally other ingredients;
b) freezing the resulting mixture of a) to obtain a frozen material;
c) packing the frozen material.

In a particular embodiment of the present invention the concentrated culture is pelletized.

In a preferred embodiment of the present invention the method for obtaining the concentrated culture of the present composition is by mixing a formulation of an amino acid, a peptide and/or a protein and a Staphylococcus specie having nitrate reductase activity. The obtained culture may be dried or frozen afterwards.

In a preferred embodiment a dried culture is obtained by
a) mixing a formulation of an amino acid, a peptide and/or a protein and a Staphylococcus specie having nitrate reductase activity and optionally other ingredients;
b) drying the resulting mixture of a) to obtain a dried material;
c) optionally grinding the obtained dried material of b); and
d) packing the dried material.

The drying may be performed by but are not limited to vacuum drying, freeze drying and/or spray drying.

In a more preferred embodiment of the present invention the freeze-dried culture is obtained by
a) mixing a formulation of an amino acid, a peptide and/or a protein and a Staphylococcus specie having nitrate reductase activity and optionally other ingredients;
b) freezing the resulting mixture of a) to obtain a frozen material;
c) subliming water from the frozen material to freeze-dry the material of b);

d) Optionally grinding the obtained freeze-dried material of c); and

e) packing the freeze-dried material.

In another preferred embodiment of the present invention a spray-dried culture is obtained by
a) mixing a formulation of an amino acid, a peptide and/or a protein and a Staphylococcus specie having nitrate reductase activity and optionally other ingredients;
b) spray drying the resulting mixture of a) to obtain a spray-dried material;
c) packing the spray-dried material.

1.

In another preferred embodiment of the present invention a frozen culture is obtained by
a) mixing a formulation of an amino acid, a peptide and/or a protein and a Staphylococcus specie having nitrate reductase activity and optionally other ingredients;
b) freezing the resulting mixture of a) to obtain a frozen material;
c) packing the frozen material.

In a particular embodiment of the present invention the concentrated culture is pelletized.

One aspect of the invention relates to a method for preserving and/or increasing the nitrate reductase activity of a bacteria having nitrate reductase activity selected from the group consisting of lactic acid bacteria and Micrococcaceae bacteria (such as e.g. a Staphylococcus specie). Thus, one embodiment of said method involves the steps of:
a) mixing a formulation comprising an amino acid, a peptide and/or a protein, and a bacterium having nitrate reductase activity selected from the group consisting of lactic acid bacteria and Micrococcaceae bacteria (such as e.g. a Staphylococcus specie) to obtain a mixture comprising an amino acid, a peptide and/or a protein and bacteria;
b) optionally freezing the resulting mixture of a) to obtain a frozen material;
c) drying the mixture of a) or the frozen material of step b) to obtain a dried material; and
d) optionally packing the frozen material obtained from step b) or the dried material of step c) to obtain a packaged product.

In a preferred embodiment, said method for preserving and/or increasing the nitrate reductase activity of a bacteria having nitrate reductase activity involves the steps of:
a) mixing a formulation comprising an amino acid, a peptide and/or a protein and a bacterium having nitrate reductase activity selected from the group consisting of lactic acid bacteria and Micrococcaceae bacteria (such as e.g. a Staphylococcus specie) to obtain a mixture comprising an amino acid, a peptide and/or a protein and bacteria;
b) freezing the resulting mixture of a) to obtain a frozen material;
c) drying the frozen material of step b) to obtain a dried material; and
d) optionally packing the dried material of step c) to obtain a packaged product.

One aspect of the invention relates to the use an amino acid, a peptide and/or a protein, for preserving and/or increasing the nitrate reductase activity of a bacteria selected from the group consisting of lactic acid bacteria and Micrococcaceae bacteria (such as a Staphylococcus specie).

In this aspect, the amino acid, a peptide and/or a protein may be defined by any of the embodiments described herein. Further, the bacteria selected from the group consisting of lactic acid bacteria and Micrococcaceae bacteria (such as a Staphylococcus specie) may selected according to any embodiment described herein.

The present invention furthermore relates to a method for reddening of a food product comprising the steps of adding the concentrated culture of the present invention to a food product, and fermenting, ripening or curing the food product with the concentrated culture.

The food product may be any product based on a food source containing myoglobin. In a preferred embodiment the food product is a meat product.

The meat product may be any product with a content of meat. The meat may be bovine meat, pork meat, poultry meat, game meat or any other category of meat.

The food product may also be a product based on fish and/or based on crustaceans.

In a preferred embodiment the concentrated culture comprising lactic acid bacteria and/or Micrococcaceae bacteria is added in a quantity of from 1.0×10⁸ to 1.0×10¹² CFU/kg, such as from 1.0×10⁹ to 1.0×10¹¹ CFU/kg lactic acid bacteria and/or a Micrococcaceae bacteria. Preferably the composition is added in a quantity of from 2.0×10⁹ to 5.0×10¹⁰ CFU/kg of lactic acid bacteria and/or a Micrococcaceae bacteria.

In a preferred embodiment the concentrated culture comprising Staphylococcus specie is added in a quantity of from 1.0×10⁸ to 1.0×10¹² CFU/kg, such as from 1.0×10⁹ to 1.0×10¹¹ CFU/kg Staphylococcus specie. Preferably the composition is added in a quantity of from 2.0×10⁹ to 5.0×10¹⁰ CFU/kg of Staphylococcus specie.

The invention is also directed to a food product obtainable by the method for reddening a food product described above or a food product obtainable by the use for reddening a food product.

### Items

The following items may further serve to describe the invention:
1. A concentrated culture of lactic acid bacteria and/or Micrococcaceae bacteria having nitrate reductase activity comprising a formulation of an amino acid, a peptide and/or a protein.
2. The concentrated culture of item 1, wherein the Micrococcaceae bacteria is a staphylococcus specie having nitrate reductase activity.
3. The concentrated culture of item 2, wherein the Staphylococcus specie is selected from the group consisting of Staphylococcus xylosus, carnosus and/or vitulinus.
4. The concentrated culture of item 1, wherein the lactic acid bacteria is a Lactobacillus pentosus having nitrate reductase activity.
5. The concentrated culture of any preceding items, wherein the amino acid is selected from the group consisting of glycine, alanine, valine, leucine, isoleucine, methionine, proline, phenylalanine, tryptophan, serine, threonine, cysteine, tyrosine, asparagine, glutamine, aspartic acid, glutamic acid, lysine, arginine and/or histidine.
6. The concentrated culture according to any of items 1 to 4, wherein the peptide is selected from the group consisting of peptides derived from yeast, soy, wheat, pea, potato, cotton seed, skimmed milk, casein, whey, meat and/or any hydrolyzed protein.
7. The concentrated culture according to any of items 1 to 4, wherein the protein is selected from the group consisting of soy protein, wheat protein, pea protein, potato protein, cotton seed protein, skimmed milk powder, sodium caseinate, whey protein, meat protein and/or any hydrolyzed protein.
8. The concentrated culture according to any of the preceding items, wherein the amino acid and/or peptide and/or protein is present in the form of a yeast extract.
9. The concentrated culture of any preceding items further comprising a monosaccharide, a disaccharide, a trisaccharide, an oligosaccharide and/or a polysaccharide.
10. The concentrated culture of item 9, wherein the disaccharide may be selected from the group consisting of sucrose, lactose, maltose and trehalose.
11. The composition of item 9, wherein the polysaccharide is maltodextrin,
12. The composition of any preceding items further comprising a citrate wherein the citrate is either citric acid or salts of citric acid.
13. The composition of any of the preceding items, wherein the concentrated culture is dry.
14. The composition of any of the preceding items, wherein the concentrated culture is a frozen, freeze dried, vacuum dried and/or spray dried composition.
15. A method for making the concentrated culture of item 1 comprising:
   a) mixing a formulation comprising an amino acid, a peptide and/or a protein and lactic acid bacteria and/or Micrococcaceae bacteria having nitrate reductase activity.
16. A method for making a dried concentrated culture comprising:
   a) mixing a formulation comprising an amino acid, a peptide and/or a protein and lactic acid bacteria and/or Micrococcaceae bacteria having nitrate reductase activity;
   b) drying the resulting mixture of a) to obtain a dried material;
   c) optionally grinding the obtained dried material of b); and
   d) packing the dried material.
17. The method of item 16, wherein the drying is performed by vacuum drying, freeze drying and/or spray drying.
18. A method for making a frozen concentrated culture comprising:
   a) mixing a formulation comprising an amino acid, a peptide and/or a protein and lactic acid bacteria and/or Micrococcaceae bacteria having nitrate reductase activity;
   b) freezing the resulting mixture of a) to obtain a frozen material;
   c) packing the frozen material.
19. A method for making a freeze-dried concentrated culture comprising:
   a) mixing a formulation comprising an amino acid, a peptide and/or a protein and lactic acid bacteria and/or Micrococcaceae bacteria having nitrate reductase activity;
   b) freezing the resulting mixture of a) to obtain a frozen material;
   c) subliming water from the frozen material to freeze-dry the material of b);
   d) Optionally grinding the obtained freeze-dried material of c); and
   e) packing the freeze-dried material.
20. A method for making a spray-dried concentrated culture comprising:
   a) mixing a formulation comprising an amino acid, a peptide and/or a protein and lactic acid bacteria and/or Micrococcaceae bacteria having nitrate reductase activity;
   b) spray drying the resulting mixture of a) to obtain a spray-dried material; and
   c) packing the spray-dried material.
21. A method for making the concentrated culture of item 2 comprising:
   a) mixing a formulation comprising an amino acid, a peptide and/or a protein and a Staphylococcus specie having nitrate reductase activity.
22. The method of any of items 15 to 21, wherein the Micrococcaceae bacteria is a Staphylococcus specie.
23. A method for making a dried concentrated culture comprising:
   a) mixing a formulation comprising an amino acid, a peptide and/or a protein and a Staphylococcus specie having nitrate reductase activity;
   b) drying the resulting mixture of a) to obtain a dried material;
   c) optionally grinding the obtained dried material of b); and
   d) packing the dried material.
24. A method for making a frozen concentrated culture comprising:
   a) mixing a formulation comprising an amino acid, a peptide and/or a protein and a Staphylococcus specie having nitrate reductase activity;
   b) freezing the resulting mixture of a) to obtain a frozen material;
   c) packing the frozen material.
25. A method for making a freeze-dried concentrated culture comprising:
   a) mixing a formulation comprising an amino acid, a peptide and/or a protein and a Staphylococcus specie having nitrate reductase activity;
   b) freezing the resulting mixture of a) to obtain a frozen material;
   c) subliming water from the frozen material to freeze-dry the material of b);
   d) Optionally grinding the obtained freeze-dried material of c); and
   e) packing the freeze-dried material.
26. A method for making a spray-dried concentrated culture comprising:
   a) mixing a formulation comprising an amino acid, a peptide and/or a protein and a Staphylococcus specie having nitrate reductase activity;
   b) spray drying the resulting mixture of a) to obtain a spray-dried material; and
   c) packing the spray-dried material.

27. The method according to items 21 to 26, wherein the Staphylococcus specie having nitrate reductase activity is a Staphylococcus vitulinus specie or a Staphylococcus carnosus specie.
28. The method according to item 25, wherein the Staphylococcus vitulinus specie is selected from the group consisting of the Staphylococcus vitulinus specie CHCC10896 that was deposited with the Deutsche Sammlung von Mikroorganismen und Zellkulturen (DSMZ) under the accession no. DSM 25789 or the Staphylococcus vitulinus specie CHCC11576 that was deposited with the Deutsche Sammlung von Mikroorganismen und Zellkulturen (DSMZ) under the accession no. DSM 27311 and mutants derived thereof.
29. The method according to any of items 15 to 28, wherein the concentrated culture is pelletized.
30. A method for reddening a food product comprising the steps of
   a) adding the composition according to any of items 1 to 14 to a meat product; and
   b) fermenting, ripening or curing the meat product with the composition.
31. The method according to item 30, wherein the food product is a meat product.
32. Use of the composition according to any of items 1 to 14 for reddening a food product.

Embodiments of the present invention are described below, by way of non-limiting examples.

### EXAMPLES

The following method can be used for determining nitrate reductase activity (NRA): The nitrite reductase activity may be determined by using the Griess test for quantification of nitrite. The method is performed as a kinetic experiment, thereby monitoring the formation of nitrite (nitrate conversion) as a function of time. Reaction rates are obtained at different cell densities.

The results are reported as nitrite formed per time unit per active cell. Number of active cells per gram dry sample is measured by flow cytometry and staining with the membrane potential sensitive dye DiOC2(3) in accordance with “ISO 19344:2015 Milk and milk products—Starter cultures, probiotics and fermented products—Quantification of lactic acid bacteria by flow cytometry”, protocol C.

To determine nitrate reductase activity, freeze-dried bacteria are suspended in sodium phosphate buffer (pH 7) in four different concentrations for each sample targeting 0.6, 1.3, 1.9 and 2.5×109 active cells/mL. Magnetic stirring bars are added to the suspensions, and a layer of mineral oil is placed on top of to protect the samples from environmental oxygen. After temperature equilibration to 45° C., glucose and nitrate in sodium phosphate buffer (pH 7) are added to the samples. After 10, 20, 30, 40, 50 and 60 minutes a small aliquot of the suspension is aspired and transferred to phosphoric acid (1 M) for nitrite concentration determination.

Griess-Ilosvay reagent (sulfanilic acid and 1-naphthylamine in acetic acid) are added to the samples diluted in phosphoric acid. After brief incubation in the dark (5-30 minutes), spectrophotometric measurement of the diazotization product of nitrite and the Griess reagents are measured (absorbance 540 nm). By using a nitrite standard curve the absorbance measured can be used to calculate the nitrite concentration in the samples investigated. The rate of nitrate conversion can then be determined for the different dilutions (cell concentrations) of a sample investigated and then in turn the rate of nitrate conversion per cell can be determined. The function of nitrite concentration formed pr. time versus cell concentration must give a coefficient of determination above 0.975 (R2>0.975, coefficient of correlation, R>0.95) to be acceptable.

### Example 1

Preparation of a freeze-dried material of Staphylococcus carnosus 15 different solutions were prepared according to Table 1.

TABLE 1
Fifteen different formulations:

| | Formulation No. | | | | | | | |
|---|---|---|---|---|---|---|---|---|
| | 1 | 2 | 3 | 4 | 5 | 6 | 7 | 8 |
| Sucrose | 4 | 4 | 4 | 4 | 4 | 4.3 | 4.1 | 4.3 |
| Maltodextrin | 9.2 | 9.2 | 9.2 | 9.2 | 9.2 | 9.7 | 9.3 | 9.8 |
| DE12 | | | | | | | | |
| Sodium | 1 | 0 | 0 | 0 | 0 | 0 | 0 | 0 |
| caseinate | | | | | | | | |
| Potato protein | 0 | 1 | 0 | 0 | 0 | 0 | 0 | 0 |
| Hydrolyzed | 0 | 0 | 1 | 0 | 0 | 0 | 0 | 0 |
| casein | | | | | | | | |
| Hydrolyzed | 0 | 0 | 0 | 1 | 0 | 0 | 0 | 0 |
| potato protein | | | | | | | | |
| Hydrolyzed | 0 | 0 | 0 | 0 | 1 | 0 | 0 | 0 |
| pea protein | | | | | | | | |
| Histidine | 0 | 0 | 0 | 0 | 0 | 0.2 | 0.8 | 0 |
| Tryptophan | 0 | 0 | 0 | 0 | 0 | 0 | 0 | 0.1 |
| Glycine | 0 | 0 | 0 | 0 | 0 | 0 | 0 | 0 |
| Phenylalanine | 0 | 0 | 0 | 0 | 0 | 0 | 0 | 0 |
| Yeast extract | 0 | 0 | 0 | 0 | 0 | 0 | 0 | 0 |
| (NuCel | | | | | | | | |
| 545 MG) | | | | | | | | |
| Water | 35.9 | 35.9 | 35.9 | 35.9 | 35.9 | 35.9 | 35.9 | 35.9 |
| Total | 50.1 | 50.1 | 50.1 | 50.1 | 50.1 | 50.1 | 50.1 | 50.1 |

| | Formulation | | | | | | |
|---|---|---|---|---|---|---|---|
| | 9 | 10 | 11 | 12 | 13 | 14 | 15 |
| Sucrose | 4.2 | 4.3 | 4.2 | 4.3 | 4 | 4 | 4.3 |
| Maltodextrin | 9.5 | 9.8 | 9.5 | 9.7 | 9.2 | 9.2 | 9.8 |
| DE12 | | | | | | | |
| Sodium | 0 | 0 | 0 | 0 | 0 | 0 | 0 |
| caseinate | | | | | | | |
| Potato protein | 0 | 0 | 0 | 0 | 0 | 0 | 0 |
| Hydrolyzed | 0 | 0 | 0 | 0 | 0 | 0 | 0 |
| casein | | | | | | | |
| Hydrolyzed | 0 | 0 | 0 | 0 | 0 | 0 | 0 |
| potato protein | | | | | | | |
| Hydrolyzed | 0 | 0 | 0 | 0 | 0 | 0 | 0 |
| pea protein | | | | | | | |
| Histidine | 0 | 0 | 0 | 0 | 0 | 0 | 0 |
| Tryptophan | 0.5 | 0 | 0 | 0 | 0 | 0 | 0 |
| Glycine | 0 | 0.1 | 0.5 | 0 | 0 | 0 | 0 |
| Phenylalanine | 0 | 0 | 0 | 0.2 | 1 | 0 | 0 |
| Yeast extract | 0 | 0 | 0 | 0 | 0 | 1 | 0 |
| (NuCel | | | | | | | |
| 545 MG) | | | | | | | |
| Water | 35.9 | 35.9 | 35.9 | 35.9 | 35.9 | 35.9 | 35.9 |
| Total | 50.1 | 50.1 | 50.1 | 50.1 | 50.1 | 50.1 | 50.1 |

From Table 1 it is seen that the amount of dry ingredients was the same for all 15 solutions as well as the dry matter of the final solutions. This means all the formulations were equal except for the 10 different peptides, proteins and amino acids to be tested.

The amino acids were tested in two different levels.

31 g of each formulation was mixed with 250 g of concentrate (Staphylococcus carnosus). After mixing the samples were kept at −50° C. before freeze drying and grinding.

The determination of the conversion rate of nitrate to nitrite (nitrate reductase activity) of a Staphylococcus specie in question was determined by measurement of the concentration of nitrite to a given time e.g. in a kinetic set-up of the Griess test (Reference to: Colorimetry and Spectrophotometry” Vogel's Textbook of Quantitative Chemical Analysis, 5th Edition. Longman. p. 702. ISBN 0-582-44693-7) as described in the Examples herein. The results are shown in Table 2.

TABLE 2
Percentage increased Nitrate conversion rate (NRA) of
Staphylococcus carnosus with different formulations
compared to a formulation without added
amino acids, peptides or proteins.

| | | Percentage increase of NRA |
|---|---|---|
| Formulation | Tested | compared to a formulation only |
| No. | compounds | comprising carbohydrates (#15) |
| 1 | Sodium | 60% |
| | Caseinate | |
| 2 | Potato protein | 56% |
| 3 | Hydrolyzed | 65% |
| | casein | |
| 4 | Hydrolyzed | 58% |
| | potato protein | |
| 5 | Hydrolyzed pea | 67% |
| | protein | |
| 6 | Histidine | 58% |
| 7 | Histidine | 71% |
| 8 | Tryptophan | 52% |
| 9 | Tryptophan | 46% |
| 10 | Glycine | 31% |
| 11 | Glycine | 54% |
| 12 | Phenylalanine | 25% |
| 13 | Phenylalanine | 27% |
| 14 | Yeast extract | 31% |

*the formulations are specified in table 1.

It can be concluded that amino acids, peptides and proteins have a significant positive effect on the NRA.

### REFERENCES

EP 0259739 MILES LABORATORIES, INC.
US 2011/008874
EP 1867713 (Kyowa Hakko)
WO 2013/186348
Colorimetry and Spectrophotometry” Vogel's Textbook of Quantitative Chemical Analysis, 5th Edition. Longman. p. 702. ISBN 0-582-44693-7
Cárcoba et al. (Eur Food Res Technol (2000) 211, 433-437, “Influence of cryoprotectants on the viability and acidifying activity of frozen and freeze-dried cells of the novel starter strain Lactococcus lactis subsp. lactis CECT 5180”
Champagne et al: The freeze-drying of . . . , Canadian Institute of food science and technology journal, vol. 24, July 1991, pages 118-128
Chavarri et al., “Cryoprotective agents for frozen concentrated starters from non-bitter Streptococcus Lactis strains”, (Biotechnology letters, vol 10, 1, 11-16 (1988)
Gøtterup et al., “Colour formation in fermented sausages by meat-associated staphylococci with different nitrite- and nitrate-reductase activities”, Meat science 2008 April; 78(4): 492-501
Mendoza et al: “Survival and beneficial properties of lactic acid bacteria from raniculture subjected to freeze-drying and storage”, Journal of applied Microbiology, Vol. 116, January 2014, pages 158-162
Mogensen et al., “Inventory of microorganisms with a documented history of use in food”, (2002) Bulletin of the IDF No. 377, 10-19

### DEPOSITS AND EXPERT SOLUTION

The applicant requests that a sample of the deposited micro-organisms stated below may only be made available to an expert, until the date on which the patent is granted.

The strain Staphylococcus vitulinus CHCC10896 that was deposited at Deutsche Sammlung von Mikroorganismen und Zellkulturen (DSMZ) GmbH, Inhoffenstr. 7B, D-38124 Braunschweig, Germany, on 15 Mar. 2012, under the accession No. DSM 25789.

The strain Staphylococcus vitulinus strain CHCC11576 has been deposited at Deutsche Sammlung von Mikroorganismen und Zellkulturen (DSMZ) GmbH, Inhoffenstr. 7B, D-38124 Braunschweig, Germany, on 11 Jun. 2013, under the accession No. DSM 27311.

The strain Staphylococcus carnosus strain CHCC4055 has been deposited at Deutsche Sammlung von Mikroorganismen und Zellkulturen (DSMZ) GmbH, Inhoffenstr. 7B, D-38124 Braunschweig, Germany, on 20 Mar. 2018, under the accession No. DSM 32779.

The strain Lactobacillus pentosus strain CHCC4196 has been deposited at Deutsche Sammlung von Mikroorganismen und Zellkulturen (DSMZ) GmbH Inhoffenstr. 7B, D-38124 Braunschweig, Germany, on 12 Jul. 2011 under the accession No. DSM 25011.

The strain Kocuria salsicia strain CHCC5184 has been deposited at Deutsche Sammlung von Mikroorganismen und Zellkulturen (DSMZ) GmbH Inhoffenstr. 7B, D-38124 Braunschweig, Germany, on 5 Jun. 2018 under the accession no. DSM 32827.

The strain Kocuria salsicia strain CHCC5185 has been deposited at Deutsche Sammlung von Mikroorganismen und Zellkulturen (DSMZ) GmbH Inhoffenstr. 7B, D-38124 Braunschweig, Germany, on 5 Jun. 2018 under the accession no. DSM 32828.

The deposits were made according to the Budapest treaty on the international recognition of the deposit of microorganisms for the purposes of patent procedure.

1. A concentrated culture comprising a bacterium having nitrate reductase activity selected from the group consisting of lactic acid bacteria and Micrococcaceae bacteria (e.g. one or more species of Staphylococcus) having nitrate reductase activity and a formulation comprising an amino acid, a peptide and/or a protein.

2. The concentrated culture of claim 1, wherein the bacteria having nitrate reductase activity is one or more species selected from the group consisting of the genera Staphylococcus and Kocuria.

3. The concentrated culture of claims 1 and 2, wherein the bacteria having nitrate reductase activity is a Kocuria specie having nitrate reductase activity.

4. The concentrated culture according to any one of the preceding claims, wherein the bacteria having nitrate reductase activity is one or more species of Staphylococcus.

5. The concentrated culture according to any one of the preceding claims, wherein the Staphylococcus specie is selected from the group consisting of Staphylococcus xylosus, carnosus and/or vitulinus.

6. The concentrated culture according to any one of the preceding claims, wherein the Staphylococcus specie is selected from the group consisting of Staphylococcus xylosus, carnosus and/or vitulinus.

7. The concentrated culture according to any one of the preceding claims, wherein the Staphylococcus specie is Staphylococcus carnosus.

8. The concentrated culture according to any one of the preceding claims, wherein the lactic acid bacteria is a Lactobacillus pentosus having nitrate reductase activity.

9. The concentrated culture according to any one of the preceding claims, comprising one or more amino acids, peptides and/or proteins selected from the group consisting of caseinate (such as sodium caseinate), potato protein, hydrolyzed casein, hydrolyzed potato protein, hydrolyzed pea protein, histidine, tryptophan, glycine, phenylalanine and yeast extract.

10. The concentrated culture according to any one of the preceding claims, wherein the amino acid is selected from the group consisting of glycine, alanine, valine, leucine, isoleucine, methionine, proline, phenylalanine, tryptophan, serine, threonine, cysteine, tyrosine, asparagine, glutamine, aspartic acid, glutamic acid, lysine, arginine and/or histidine.

11. The concentrated culture according to any one of the preceding claims, wherein the amino acid is one or more amino acids selected from the group consisting of histidine, tryptophan, glycine and phenylalanine.

12. The concentrated culture according to any one of the preceding claims, wherein the amino acid is histidine.

13. The concentrated culture according to any one of the preceding claims, wherein the amino acid is tryptophan.

14. The concentrated culture according to any one of the preceding claims, wherein the amino acid is glycine.

15. The concentrated culture according to any one of the preceding claims, wherein the amino acid is phenylalanine.

16. The concentrated culture according to any one of the preceding claims, wherein the peptide is selected from the group consisting of peptides derived from yeast, soy, wheat, pea, potato, cotton seed, skimmed milk, casein, whey, meat and/or any hydrolyzed protein.

17. The concentrated culture according to any one of the preceding claims, wherein the peptide is selected from the group consisting of peptides derived from yeast, soy, wheat, pea, potato, cotton seed, skimmed milk, casein, whey, meat and/or any hydrolyzed protein.

18. The concentrated culture according to any one of the preceding claims, wherein the formulation comprises a hydrolyzed protein selected from the group consisting of hydrolyzed casein, hydrolyzed potato protein and hydrolyzed pea protein.

19. The concentrated culture according to any one of the preceding claims, wherein the formulation comprises hydrolyzed casein.

20. The concentrated culture according to any one of the preceding claims, wherein the formulation comprises hydrolyzed potato protein.

21. The concentrated culture according to any one of the preceding claims, wherein the formulation comprises hydrolyzed pea protein.

22. The concentrated culture according to any one of the preceding claims, wherein the formulation does not comprise gelatin.

23. The concentrated culture according to any one of the preceding claims, wherein the peptide is selected from the group consisting of hydrolyzed casein, hydrolyzed potato protein, hydrolyzed pea protein and yeast extract.

24. The concentrated culture according to any one of the preceding claims, wherein the peptide is yeast extract.

25. The concentrated culture according to any one of the preceding claims, wherein the protein is selected from the group consisting of casein or caseinate, potato protein, and pea protein.

26. The concentrated culture according to any one of the preceding claims, wherein the protein is casein or caseinate.

27. The concentrated culture according to any one of the preceding claims, wherein the protein is potato protein.

28. The concentrated culture according to any one of the preceding claims, wherein the protein is pea protein.

29. The concentrated culture according to any one of the preceding claims, wherein the protein is selected from the group consisting of soy protein, wheat protein, pea protein, potato protein, cotton seed protein, skimmed milk powder, sodium caseinate, whey protein, meat protein and/or any hydrolyzed protein.

30. The concentrated culture according to any of the preceding claims, wherein the amino acid and/or peptide and/or protein is present in the form of a yeast extract.

31. The concentrated culture according to any one of the preceding claims, further comprising a monosaccharide, a disaccharide, a trisaccharide, an oligosaccharide and/or a polysaccharide.

32. The concentrated culture according to any one of the preceding claims, wherein the disaccharide may be selected from the group consisting of sucrose, lactose, maltose and trehalose.

33. The concentrated culture according to any one of the preceding claims, further comprising sucrose.

34. The concentrated culture according to any one of the preceding claims, further comprising maltodextrin, such as maltodextrin DE 3 to 20 (e.g. maltodextrin DE 12).

35. The concentrated culture according to any one of the preceding claims, wherein said concentrated culture comprises or consists of one or more species of Staphylococcaceae (e.g. one or more species of Staphylococcus), an amino acid, a peptide and/or protein, a disaccharide (e.g. sucrose), and a polysaccharide (e.g. maltodextrin), and optionally water.

36. The concentrated culture according to any one of the preceding claims, wherein said concentrated culture comprises or consists of one or more species of Staphylococcaceae (e.g. one or more species of Staphylococcus), an amino acid, a peptide and/or protein, sucrose, and maltodextrin (e.g. maltodextrin DE 12), and optionally water.

37. The concentrated culture according to any one of the preceding claims further comprising a sugar alcohol or a derivative thereof.

38. The concentrated culture according to any one of the preceding claims further comprising inositol or a derivative thereof (e.g. form of inositol phosphate).

39. The concentrated culture according to any one of the preceding claims, further comprising a citrate wherein the citrate is either citric acid or salts of citric acid.

40. The concentrated culture according to any one of the preceding claims, wherein the concentrated culture is dry and/or dried, preferably freeze dried.

41. The concentrated culture according to any one of the preceding claims, wherein the concentrated culture is a frozen, freeze dried, vacuum dried and/or spray-dried composition.

42. The concentrated culture according to any one of the preceding claims, wherein the Staphylococcus specie having nitrate reductase activity is a Staphylococcus vitulinus specie or a Staphylococcus carnosus specie.

43. The concentrated culture according to any one of the preceding claims, wherein the Staphylococcus specie is selected from the group consisting of Staphylococcus carnosus strain CHCC4055 deposited with the Deutsche Sammlung von Mikroorganismen und Zellkulturen (DSMZ) under the accession no. DSM 32779 and mutants derived thereof; the Staphylococcus vitulinus specie CHCC10896 deposited with the Deutsche Sammlung von Mikroorganismen und Zellkulturen (DSMZ) under the accession no. DSM 25789; and the Staphylococcus vitulinus specie CHCC11576 deposited with the Deutsche Sammlung von Mikroorganismen und Zellkulturen (DSMZ) under the accession no. DSM 27311 and mutants derived thereof.

44. The concentrated culture according to any one of the preceding claims, wherein the Staphylococcus specie is selected from the group consisting of the Staphylococcus vitulinus specie CHCC10896 that was deposited with the Deutsche Sammlung von Mikroorganismen und Zellkulturen (DSMZ) under the accession no. DSM 25789 or the Staphylococcus vitulinus specie CHCC11576 that was deposited with the Deutsche Sammlung von Mikroorganismen und Zellkulturen (DSMZ) under the accession no. DSM 27311 and mutants derived thereof.

45. The concentrated culture according to any one of the preceding claims, wherein the Staphylococcus specie is Staphylococcus vitulinus specie CHCC10896 deposited with the Deutsche Sammlung von Mikroorganismen und Zellkulturen (DSMZ) under the accession no. DSM 25789.

46. The concentrated culture according to any one of the preceding claims, wherein the Staphylococcus specie is Staphylococcus vitulinus specie CHCC11576 deposited with the Deutsche Sammlung von Mikroorganismen und Zellkulturen (DSMZ) under the accession no. DSM 27311 and mutants derived thereof.

47. The concentrated culture according to any one of the preceding claims, wherein the Staphylococcus specie is selected from the group consisting of Staphylococcus carnosus strain CHCC4055 deposited with the Deutsche Sammlung von Mikroorganismen und Zellkulturen (DSMZ) under the accession no. DSM 32779 and mutants derived thereof.

48. Use an amino acid, a peptide and/or a protein, for preserving and/or increasing the nitrate reductase activity of a bacteria selected from the group consisting of lactic acid bacteria and Micrococcaceae bacteria (such as a Staphylococcus specie).

49. The use according to claim 48 for preserving and/or increasing the nitrate reductase activity of one or more Staphylococcus species selected from the group consisting of Staphylococcus xylosus, Staphylococcus carnosus and Staphylococcus vitulinus.

50. The use according to any one of claims 48 to 49 for preserving and/or increasing the nitrate reductase activity of one or more Staphylococcus species selected from the group consisting of Staphylococcus carnosus and Staphylococcus vitulinus.

51. The use according to any one of claims 48 to 50, wherein the bacteria (such as a Staphylococcus species) is comprised in a concentrated culture as defined by claims 1 to 47.

52. The use according to any one of claims 48 to 51, wherein the bacteria (such as a Staphylococcus species) is comprised in a concentrated culture produced by the method of any one of claims 53 to 87.

53. A method for preserving and/or increasing the nitrate reductase activity of a bacteria having nitrate reductase activity selected from the group consisting of lactic acid bacteria and Micrococcaceae bacteria (such as e.g. a Staphylococcus strain) having nitrate reductase activity comprising the steps of:
a) mixing a formulation comprising an amino acid, a peptide and/or a protein and lactic acid bacteria and/or Micrococcaceae bacteria (such as e.g. a Staphylococcus specie) having nitrate reductase activity to obtain a mixture comprising an amino acid, a peptide and/or a protein and bacteria;
b) optionally freezing the resulting mixture of a) to obtain a frozen material;
c) drying the mixture of a) or the frozen material of step b) to obtain a dried material; and
d) optionally packing the frozen material obtained from step b) or the dried material of step c) to obtain a packaged product.

54. The method according to claim 53, wherein the resulting mixture of a) is frozen to obtain a frozen material as defined in step b).

55. The method according to claims 53 to 54, wherein the mixture of a), or the frozen material of step b), is dried to obtain a dried material.

56. The method according to claims 53 to 55, wherein the mixture of a), or the frozen material of step b), is dried by desiccation, fluidized bed drying, freeze drying, vacuum drying and/or spray drying, preferably by freeze drying.

57. The method according to claims 53 to 56, wherein the frozen material of step b), is dried by freeze drying and/or freeze drying.

58. The method according to any one of claims 53 to 57 wherein the bacteria having nitrate reductase activity is selected from the group consisting of Staphylococcaceae bacteria, such as species of the genus Staphylococcus.

59. The method according to any one of claims 53 to 58, wherein the bacteria having nitrate reductase activity is one or more Staphylococcus species selected from the group consisting of Staphylococcus xylosus, Staphylococcus carnosus and Staphylococcus vitulinus.

60. The method according to any one of claims 53 to 59, wherein the end-product of the method is a concentrated culture as defined by any one of claims 1 to 47.

61. A method for making the concentrated culture as defined by claims 1 to 47 comprising:
a) mixing a formulation comprising an amino acid, a peptide and/or a protein and lactic acid bacteria and/or Micrococcaceae bacteria (e.g. one or more a species of Staphylococcus) having nitrate reductase activity.

62. A method for making a dried concentrated culture comprising:
a) mixing a formulation comprising an amino acid, a peptide and/or a protein and lactic acid bacteria and/or Micrococcaceae bacteria (e.g. one or more a species of Staphylococcus) having nitrate reductase activity;
b) drying the resulting mixture of a) to obtain a dried material;
c) optionally grinding the obtained dried material of b); and
d) packing the dried material.

63. The method of claim 62, wherein the drying is performed by vacuum drying, freeze drying and/or spray drying.

64. A method for making a frozen concentrated culture comprising:
a) mixing a formulation comprising an amino acid, a peptide and/or a protein and lactic acid bacteria and/or Micrococcaceae bacteria (e.g. one or more a species of Staphylococcus) having nitrate reductase activity;
b) freezing the resulting mixture of a) to obtain a frozen material;
c) packing the frozen material.

65. A method for making a freeze-dried concentrated culture comprising:
a) mixing a formulation comprising an amino acid, a peptide and/or a protein and lactic acid bacteria and/or Micrococcaceae bacteria (e.g. one or more a species of Staphylococcus) having nitrate reductase activity;
b) freezing the resulting mixture of a) to obtain a frozen material;
c) subliming water from the frozen material to freeze-dry the material of b);
d) Optionally grinding the obtained freeze-dried material of c); and
e) packing the freeze-dried material.

66. A method for making a spray-dried concentrated culture comprising:
a) mixing a formulation comprising an amino acid, a peptide and/or a protein and lactic acid bacteria and/or Micrococcaceae bacteria (e.g. one or more a species of Staphylococcus) having nitrate reductase activity;
b) spray drying the resulting mixture of a) to obtain a spray-dried material; and
c) packing the spray-dried material.

67. A method for making the concentrated culture as defined in claims 1 to 47 comprising:
a) mixing a formulation comprising an amino acid, a peptide and/or a protein and a Staphylococcus specie having nitrate reductase activity.

68. The method of any of claims 61 to 67, wherein the Micrococcaceae bacteria is a Kocuria specie, e.g. a Kocuria salsicia specie.

69. A method for making a dried concentrated culture comprising:
a) mixing a formulation comprising an amino acid, a peptide and/or a protein and a Staphylococcus specie having nitrate reductase activity;
b) drying the resulting mixture of a) to obtain a dried material;
c) optionally grinding the obtained dried material of b); and
d) packing the dried material.

70. A method for making a frozen concentrated culture comprising:
a) mixing a formulation comprising an amino acid, a peptide and/or a protein and a Staphylococcus specie having nitrate reductase activity;
b) freezing the resulting mixture of a) to obtain a frozen material;
c) packing the frozen material.

71. A method for making a freeze-dried concentrated culture comprising:
a) mixing a formulation comprising an amino acid, a peptide and/or a protein and a Staphylococcus specie having nitrate reductase activity;
b) freezing the resulting mixture of a) to obtain a frozen material;
c) subliming water from the frozen material to freeze-dry the material of b);
d) Optionally grinding the obtained freeze-dried material of c); and
e) packing the freeze-dried material.

72. A method for making a spray-dried concentrated culture comprising:
a) mixing a formulation comprising an amino acid, a peptide and/or a protein and a Staphylococcus specie having nitrate reductase activity;
b) spray drying the resulting mixture of a) to obtain a spray-dried material; and
c) packing the spray-dried material.

73. The method according to any one of the preceding claims, wherein the Staphylococcus specie having nitrate reductase activity is a Staphylococcus vitulinus specie or a Staphylococcus carnosus specie.

74. The method according to any one of the preceding claims, wherein the Staphylococcus specie is selected from the group consisting of Staphylococcus carnosus strain CHCC4055 deposited with the Deutsche Sammlung von Mikroorganismen und Zellkulturen (DSMZ) under the accession no. DSM 32779 and mutants derived thereof; the Staphylococcus vitulinus specie CHCC10896 deposited with the Deutsche Sammlung von Mikroorganismen und Zellkulturen (DSMZ) under the accession no. DSM 25789; and the Staphylococcus vitulinus specie CHCC11576 deposited with the Deutsche Sammlung von Mikroorganismen und Zellkulturen (DSMZ) under the accession no. DSM 27311 and mutants derived thereof.

75. The method according to any one of the preceding claims, wherein the Staphylococcus specie is selected from the group consisting of the Staphylococcus vitulinus specie CHCC10896 that was deposited with the Deutsche Sammlung von Mikroorganismen und Zellkulturen (DSMZ) under the accession no. DSM 25789 or the Staphylococcus vitulinus specie CHCC11576 that was deposited with the Deutsche Sammlung von Mikroorganismen und Zellkulturen (DSMZ) under the accession no. DSM 27311 and mutants derived thereof.

76. The method according to any one of the preceding claims, wherein the Staphylococcus specie is Staphylococcus vitulinus specie CHCC10896 deposited with the Deutsche Sammlung von Mikroorganismen und Zellkulturen (DSMZ) under the accession no. DSM 25789.

77. The method according to any one of the preceding claims, wherein the Staphylococcus specie is Staphylococcus vitulinus specie CHCC11576 deposited with the Deutsche Sammlung von Mikroorganismen und Zellkulturen (DSMZ) under the accession no. DSM 27311 and mutants derived thereof.

78. The method according to any one of the preceding claims, wherein the Staphylococcus specie is selected from the group consisting of Staphylococcus carnosus strain CHCC4055 deposited with the Deutsche Sam mlung von Mikroorganismen and Zellkulturen (DSMZ) under the accession no. DSM 32779 and mutants derived thereof.

79. The method according to any one of the preceding claims, wherein the concentrated culture is pelletized.

80. The method according to any one of the preceding claims, wherein the concentrated culture is frozen in a tray and freeze-dried.

81. The method according to any one of the preceding claims, wherein the concentrated culture is frozen in a tray, freeze-dried and ground.

82. A method for reddening a food product comprising the steps of
a) adding the concentrated culture according to any of claims 1 to 47 to a meat product; and
b) fermenting, ripening or curing the meat product with the composition.

83. The method according to claim 82, wherein the food product is a meat product.

84. Use of the composition according to any of claims 1 to 47 for reddening a food product.
